(19) United States
(12) Patent Application Publication
(10) Pub. No.: US 2008/0057499 A1
(43) Pub. Date: Mar. 6, 2008

(54) Methods for high specificity whole genome amplification and hybridization

(75) Inventor: Glenn Fu, Dublin, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, Inc., Santa Clara, CA (US)

(21) Appl. No.: 11/672,034

(22) Filed: Feb. 6, 2007

Related U.S. Application Data
(60) Provisional application No. 60/765,958, filed on Feb. 6, 2006.
(60) Provisional application No. 60/804,092, filed on Jun. 7, 2006.

(51) Int. Cl. C12Q 1/68; C12P 19/34

(52) U.S. Cl. 435/6; 435/91.2

(57) ABSTRACT

Methods for amplifying genomic DNA using semi-random primers that consist of different combinations of two non-complementary bases are disclosed. In a preferred aspect all of the primers in the collection are composed entirely of the same two non-complementary bases. In preferred aspects the DNA is amplified using R₆, Y₆, M₆ and K₆. The amplification is by a strand displacing polymerase and the amplification product may be hybridized to a high complexity array of probes without further complexity reduction. In some aspects, additives are included in the hybridization to reduce non-specific hybridization. The hybridization pattern obtained is preferably analyzed for allele specific hybridization to determine genotype. The primers in the collection are selected so that there is a minimum of self complementarity between any two primers in the collection, minimizing the occurrence of hybrids between primers.

### RELATED APPLICATIONS

This application claims priority to provisional application Nos. 60/765,958 filed Feb. 6, 2006 and 60/804,092 filed Jun. 7, 2006, both of which are incorporated herein by reference in their entireties.

### FIELD OF THE INVENTION

The invention is related to methods of amplification and analysis of complex nucleic acid samples.

### BACKGROUND OF THE INVENTION

Several methods now exist for the DNA amplification of entire genomes. The genomic DNA sample is usually amplified in a single reaction using various polymerases, often in conjunction with specific or random DNA oligonucleotides (or primers). The whole genome amplification (or WGA) procedure is often carried out using the following techniques, for example: PCR amplification, clone amplification, Multiple strand displacement amplification, lone-linker PCR, linker-adaptor PCR, degenerate oligonucleotide PCR (DOP), or T7 RNA amplification, (see also US20040209298 for a discussion of amplification methods).

More recently, with advances in DNA research technology, and with many research centers acquiring, processing or archiving large numbers of clinical samples, the need to perform WGA has grown significantly. This growth is further fueled by the difficulty in isolating large-enough quantities of DNA samples for research. Currently, there are 2 predominant kits commercially available to researchers for WGA. Sigma-Aldrich (St. Louis, Mo.) manufactures and sells the “GenomePlex WGA” kit from Rubicon genomics (Ann Arbor, Mich.). The other product on the market (Repli-G WGA kit) currently is provided by Qiagen, Inc. (Valencia, Calif.).

Currently available methods for amplification using primers of random sequence are limited in their ability to produce accurate representations of output amplified DNA. Specifically, the methods produce whole genome amplified DNA that not only originates from the input DNA that is put into the reactions, but also a significant amount of random, non-specific DNA, that does not originate from nor represent the input DNA targeted for amplification. The non-specific amplification products may result from regions of complementarity between primers. The complementary primers can hybridize generating overhanging ends and the ends can be filled in by the polymerase. Subsequent rounds of hybridization of these primer templated extension products can result in long concatamers of primer sequence, derived primarily from primer sequences. This primer based amplification can occur in the absence of added template DNA. These concatamers add to the complexity of the overall amplification product. The presence of non-specific amplification products can interfere with downstream analysis of the amplification product, for example, applications, such as hybridization of the amplified DNA to an array, such as a GeneChip microarray may suffer from inconsistencies and increased variability due to the extraneous non-specific DNA. Thus an improvement to the method to increase the specificity of the amplification procedure to result in reduced amounts of non-specific amplification products or “junk output DNA” would be beneficial for a variety of applications including hybridization to a microarray.

### SUMMARY OF THE INVENTION

Disclosed are compositions and methods for amplification of nucleic acid sequences of interest. In a preferred aspect a complex sample, such as a genomic DNA sample, is amplified using semi-random primers. The primers in the semi-random primers vary in sequence but all are composed entirely of the same two non-complementary bases. Semi random primers may be all Gs and As, Cs and Ts, As and Cs or Gs and Ts. In some embodiments the primers have uracil replacing thymine at some or all positions.

In another aspect a complex DNA sample is amplified using random RNA primers. The random RNA primers may include all possible bases. The RNA primers can form dimers, but the dimers will not be extended by a DNA dependent polymerase.

In preferred aspects the amplification product is hybridized to an array of oligonucleotide probes. The array preferably has more than 100,000 different, probes in known or determinable locations. In a preferred aspect the array is an array of allele specific probes. The probes may differ by a single base and the hybridization conditions are selected to allow for allele specific hybridization. The array may also be a collection of beads.

### BRIEF DESCRIPTION OF THE DRAWINGS

FIG. 1 shows a flowchart for screening SNPs to identify a set that performs with whole genome hybridization.

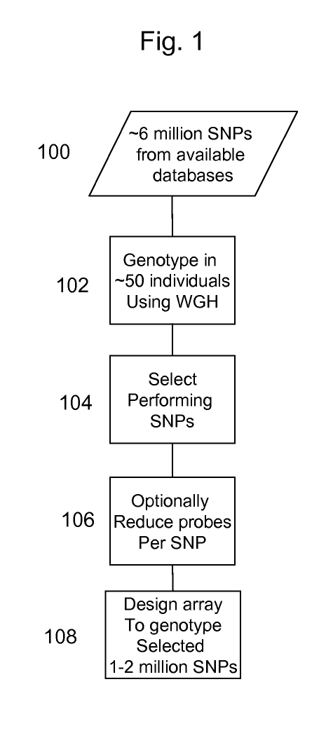

FIG. 2 shows a schematic of a method of reducing background by mismatch cleavage.

FIG. 3 shows an image of a gel showing the results of amplification using phi29 enzymes from two different sources primed with random or semi-random primers.

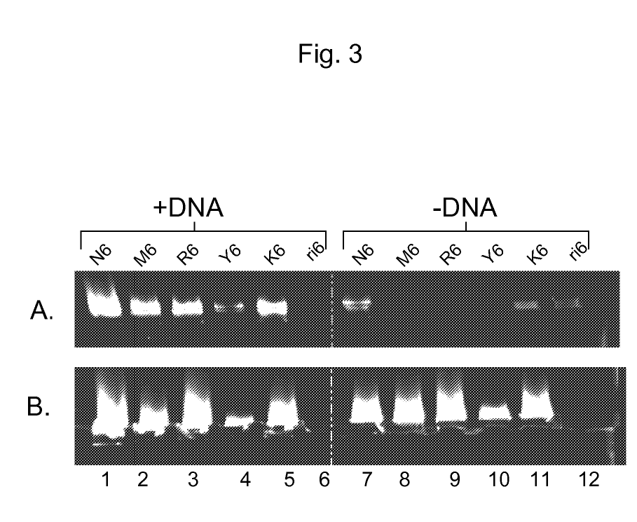

### DETAILED DESCRIPTION OF THE INVENTION

(A) General

The present invention has many preferred embodiments and relies on many patents, applications and other references for details known to those of the art. Therefore, when a patent, application, published patent application or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above. Convenient sources of genomic DNA include, for example, whole blood, semen, saliva, tears, urine, fecal material, sweat, buccal cells, skin, hair, amniotic fluid and tissue. The source of the sample may be, for example, from a tumor and may be, for example, a needle aspiration biopsy. The nucleic acid may be purified from the source or it may be analyzed as a crude cell lysate.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. All references to the function log default to e as the base (natural log) unless stated otherwise (such as log₁₀).

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in US Patent Pub. No. 20050074787, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. patent application Ser. No. 10/442,021, U.S. Patent Publication No. 20030036069 and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NASBA). (See, U.S. Pat. Nos. 5,409,818, 5,554,517, and 6,063,603, each of which is incorporated herein by reference). Other amplification methods that may be used include: Qbeta Replicase, described in PCT Patent Application No. PCT/US87/00880, isothermal amplification methods such as SDA, described in Walker et al. 1992, Nucleic Acids Res. 20(7):1691-6, 1992, and rolling circle amplification, described in U.S. Pat. No. 5,648,245. Other amplification methods that may be used are described in, U.S. Pat. Nos. 4,988,617, 5,242,794, 5,494,810, and 6,582,938 and US Pub. No. 20030143599, each of which is incorporated herein by reference. In some embodiments DNA is amplified by multiplex locus-specific PCR. In a preferred embodiment the DNA is amplified using adaptor-ligation and single primer PCR. Other available methods of amplification, such as balanced PCR (Makrigiorgos, et al. (2002), Nat Biotechnol, Vol. 20, pp. 936-9), may also be used.

Additional methods of sample preparation and techniques for reducing the complexity of a nucleic sample are described in Dong et al., Genome Research 11, 1418 (2001), in U.S. Pat. Nos. 6,361,947, 6,391,592 and 6,872,529 and U.S. Patent Publication Nos. 20030036069, 20030096235 and 20030082543. Additional methods of using a genotyping array are disclosed, for example, in US Patent Application No. 10/442,021, and U.S. Patent Publication Nos. 20040146883, 20030186280, 20030186279, 20040067493, 20030232353, 20060292597, 20050233354, 20050074799 and 20040185475.

Methods are disclosed for identifying chromosomal gains and losses at high resolution using high-density microarray genotyping methods such as whole genome sampling analysis (WGSA) (see, Kennedy et al. (2003), Nat Biotechnol, Vol., pp. 1233-1237, U.S. Pat. No. 6,361,947, U.S. Patent Publication Nos. 20030025075, 20020142314, 20040146890, 20030186279, 20040072217, 20030186280, and 20040067493 and U.S. Patent Application No. 10/442,021). WGSA simultaneously genotypes more than 10,000 SNPs in parallel by allele-specific hybridization to perfect match (PM) and mismatch (MM) probes synthesized on an array. Methods for chromosomal copy number analysis using the Affymetrix Mapping 10K array in combination with WGSA, have also been reported in Bignell et al. Genome Res. 14:287-295 (2004) and Huang et al., Hum Genomics 1:287-299 (2004). Similar analysis using the Affymetrix Mapping 100K array has also been reported in Slater et al., Am J. Hum Genet. 77:709-726 (2005).

The methods may be combined with other methods of genome analysis and complexity reduction. Other methods of complexity reduction include, for example, AFLP, see U.S. Pat. No. 6,045,994, which is incorporated herein by reference, and arbitrarily primed-PCR (AP-PCR) see McClelland and Welsh, in PCR Primer: A laboratory Manual, (1995) eds. C. Dieffenbach and G. Dveksler, Cold Spring Harbor Lab Press, for example, at p 203, which is incorporated herein by reference in its entirety. Additional methods of sample preparation and techniques for reducing the complexity of a nucleic sample are described in Dong et al., Genome Research 11, 1418 (2001), in U.S. Pat. No 6,361,947, 6,391,592, 6,458,530 and U.S. Patent Publication Nos. 20030039069, 20050079536, 20030096235, 20030082543, 20040072217, 20050142577, 20050233354, 20050227244, 20050208555, 20050074799, 20050042654 and 20040067493, which are incorporated herein by reference in their entireties.

The design and use of allele-specific probes for analyzing polymorphisms is described by e.g., Saiki et al., Nature 324, 163-166 (1986); Dattagupta, EP 235,726, and WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles.

Methods for determining copy number using high density SNP genotyping arrays using the Affymetrix 10K SNP genotyping array and the 100K Mapping Set are disclosed. The methods should also be useful for estimating copy number along with a higher density genotyping array, such as the 500K Mapping Set. The 10K array and the 100K array set use a WGSA target preparation scheme in which single primer PCR amplification of specific fractions of the genome is carried out. The 100K WGSA method uses two separate restriction enzymes that each generates a complexity fraction estimated to be about 300 Mb. The 10K array uses a single restriction enzyme and generates a sample with less than 300 Mb complexity. Both arrays have been shown to genotype SNPs, with call rates, reproducibility, and accuracy greater than 99%, 99.7%, and 99.7% respectively (Matsuzaki et al. Nat Methods 1: 109-111, 2004).

The term “WGSA (Whole Genome Sampling Assay) Genotyping Technology” refers to a technology that allows the genotyping of thousands of SNPs simultaneously in complex DNA without the use of locus-specific primers. WGSA reduces the complexity of a nucleic acid sample by amplifying a subset of the fragments in the sample. In this technique, a nucleic acid sample is fragmented with one or more restriction enzyme of interest and adaptors are ligated to the digested fragments. A single primer that is complementary of the adaptor sequence is used to amplify fragments of a desired size, for example, 400-800, 400-2000 bps, using PCR. Fragments that are outside the selected size range are not efficiently amplified. The processed target is then hybridized to nucleic acid arrays comprising SNP-containing fragments/probes. WGSA is disclosed in, for example, U.S. Patent Publication Nos. 20040185475, 20040157243 (also PCT Application published as WO04/044225), 20040146890, 20030186279, 20030186280, 20030232353, and 20040067493, and U.S. patent application Ser. Nos. 10/442,021 and 10/646,674, each of which is hereby incorporated by reference in its entirety for all purposes.

Given the millions of SNPs that are estimated to exist and the large subset already in databases, there is a need to prune this number down to a number that will fit on a few microarrays at current feature sizes. Applications of microarray for SNP genotyping have been described in e.g., a number of U.S. patents and patent applications, including U.S. Pat. Nos 6,300,063, 6,361,947, 6,368,799 U.S. patent application Ser. No. 10/442,021 and US Patent Publication Nos. 20040067493, 20030232353, 20030186279, 20050260628, and 20030186280, all incorporated herein by reference in their entireties for all purposes. Methods and arrays for simultaneous genotyping of more than 10,000 and more than 100,000 SNPs have also been described for example in Kennedy et al. (2003) Nat. Biotech. 21:1233-7, Matsuzaki et al., (2004) Genome Res. 14(3): 414-425, and Matsuzaki et al (2004) Nature Methods, Vol 1, 109-111, all incorporated herein by reference in their entireties for all purposes.

Additional methods of sample preparation and techniques for reducing the complexity of a nucleic sample are described in Dong et al., Genome Research 11, 1418 (2001), in U.S. Pat. No. 6,361,947, 6,391,592 and U.S. Patent Pub. Nos. 20030096235, 20030082543 and 20030036069.

In preferred embodiments large scale mapping of disease loci may be performed using a fixed panel of SNPs that interrogate the entire genome at a selected resolution. Arrays capable of interrogating fixed SNP panels are available from Affymetrix and include, for example, the Mapping 10K array, the Mapping 100K array set (includes 2 50K arrays) and the Mapping 500K array set (includes two ˜250K arrays). These arrays and array sets interrogate more than 10,000, 100,000 and 500,000 different human SNPs, respectively. The perfect match probes on the array are perfectly complementary to one or the other allele of a biallelic SNP. Each SNP is interrogated by a probe set comprising 24 to 40 probes. The perfect match probes in a probe set are each different, varying in, for example, the SNP allele, the position of the SNP relative to the center of the probe and the strand targeted. The probes are present in perfect match-mismatch pairs. The SNPs interrogated by a mapping array or array set are spaced throughout the genome with approximately equal spacing, for example, the SNPs in the 10K array are separated by about 200,000 base pairs. The median physical distance between SNPs in the 500K array set is 2.5 kb and the average distance between SNPs is 5.8 kb. The mean and median distance between SNPs will vary depending on the density of SNPs interrogated. Methods for using mapping arrays see, for example, Kennedy et al., Nat. Biotech. 21:1233-1237 (2003), Matsuzaki et al., Genome Res. 14:414-425 (2004), Matsuzaki et al., Nat. Meth. 1: 109-111 (2004) and U.S. Patent Pub. Nos. 20040146890 and 20050042654. Selected panels of SNPs can also be interrogated using a panel of locus specific probes in combination with a universal array as described in Hardenbol et al., Genome Res. 15:269-275 (2005) and in U.S. Pat. No. 6,858,412. Universal tag arrays and reagent kits for performing such locus specific genotyping using panels of custom molecular inversion probes (MIPs) are available from Affymetrix.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832, 5,631,734, 5,834,758, 5,936,324, 5,981,956, 6,025,601, 6,141,096, 6,185,030, 6,201,639, 6,218,803, and 6,225,625 in U.S. Patent Pub. No. 20040012676 and in PCT Application PCT/US99/06097 (published as WO99/47964), each of which also is hereby incorporated by reference in its entirety for all purposes.

In preferred aspects, the nucleic acids are labeled with a detectable label. The label may be directly or indirectly detectable. Labels, include for example, fluorescein isothiocyanate, 5,6-carboxymethyl fluorescein, Texas Red, coumarin, dansyl chloride, rhodamine, amino-methyl coumarin, Eosin, Erythrosin, BODIPY® dyes (Invitrogen), CASCADE BLUE® dye (Invitrogen), OREGON GREEN® dye (Invitrogen), ALEXA FLOUR® conjugates (Invitrogen), pyrene, lissamine, xanthene, acridine, oxazines, phycoerythrin, cyanine dyes (Cy3, Cy3.5, Cy5, Cy 5.5, Cy7), DNP or a combination thereof. Many dyes and labels and information about uses of particular labes are available from Invitrogen, Corp (Carlsbad, Calif.) and from the Invitrogen website.

Methods and apparatus for signal detection and processing of intensity data are disclosed in, for example, U.S. Pat. Nos. 5,143,854, 5,547,839, 5,578,832, 5,631,734, 5,800,992, 5,834,758, 5,856,092, 5,902,723, 5,936,324, 5,981,956, 6,025,601, 6,090,555, 6,141,096, 6,185,030, 6,201,639; 6,218,803; and 6,225,625, in U.S. Patent Pub. Nos. 20040012676 and 20050059062 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al, Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See also U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Computer methods related to genotyping using high density microarray analysis may also be used in the present methods, see, for example, US Patent Pub. Nos. 20050250151, 20050244883, 20050108197, 20050079536 and 20050042654.

Computer implemented methods for determining genotype using data from mapping arrays are disclosed, for example, in Liu, et al., Bioinformatics 19:2397-2403 (2003), Rabbee and Speed, Bioinformatics, 22:7-12 (2006), and Di et al., Bioinformatics 21:1958-63 (2005). Computer implemented methods for linkage analysis using mapping array data are disclosed, for example, in Ruschendorf and Numberg, Bioinformatics 21:2123-5 (2005) and Leykin et al, BMC Genet. 6:7, (2005). Computer methods for analysis of genotyping data are also disclosed in U.S. Patent Pub. Nos. 20060229823, 20050009069, 20040138821, 20060024715, 20050250151 and 20030009292.

Methods for analyzing chromosomal copy number using mapping arrays are disclosed, for example, in Bignell et al., Genome Res. 14:287-95 (2004), Lieberfarb, et al., Cancer Res. 63:4781-4785 (2003), Zhao et al., Cancer Res. 64:3060-71 (2004), Nannya et al., Cancer Res. 65:6071-6079 (2005) and Ishikawa et al., Biochem. and Biophys. Res. Comm., 333:1309-1314 (2005). Computer implemented methods for estimation of copy number based on hybridization intensity are disclosed in U.S. Patent Pub. Nos. 20040157243, 20050064476, 20050130217, 20060035258, 20060134674 and 20060194243.

Additionally, the present invention may have preferred embodiments that include methods for providing genetic information over networks such as the Internet as shown in U.S. Patent Pub. Nos. 20030097222, 20020183936, 20030100995, 20030120432, 20040002818, 20040126840, and 20040049354.

(B) Definitions

Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An oligonucleotide or polynucleotide is a nucleic acid ranging from at least 2, preferably at least 8, 15 or 20 nucleotides in length, but may be up to 50, 100, 1000, or 5000 nucleotides long or a compound that specifically hybridizes to a polynucleotide. Polynucleotides of the present invention include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term fragment refers to a portion of a larger DNA polynucleotide or DNA. A polynucleotide, for example, can be broken up, or fragmented into, a plurality of fragments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3^{rd} Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

“Genome” designates or denotes the complete, single-copy set of genetic instructions for an organism as coded into the DNA of the organism. A genome may be multi-chromosomal such that the DNA is cellularly distributed among a plurality of individual chromosomes. For example, in human there are 22 pairs of chromosomes plus a gender associated XX or XY pair.

The term “chromosome” refers to the heredity-bearing gene carrier of a living cell which is derived from chromatin and which comprises DNA and protein components (especially histones). The conventional internationally recognized individual human genome chromosome numbering system is employed herein. The size of an individual chromosome can vary from one type to another with a given multi-chromosomal genome and from one genome to another. In the case of the human genome, the entire DNA mass of a given chromosome is usually greater than about 100,000,000 bp. For example, the size of the entire human genome is about 3×10⁹ bp. The largest chromosome, chromosome no. 1, contains about 2.4×10⁸ bp while the smallest chromosome, chromosome no. 22, contains about 5.3×10⁷ bp.

A “chromosomal region” is a portion of a chromosome. The actual physical size or extent of any individual chromosomal region can vary greatly. The term “region” is not necessarily definitive of a particular one or more genes because a region need not take into specific account the particular coding segments (exons) of an individual gene.

The term subset or representative subset refers to a fraction of a genome. The subset may be 0.1, 1, 3, 5, 10, 25, 50 or 75% of the genome. The partitioning of fragments into subsets may be done according to a variety of physical characteristics of individual fragments. For example, fragments may be divided into subsets according to size, according to the particular combination of restriction sites at the ends of the fragment, or based on the presence or absence of one or more particular sequences.

An “array” comprises a support, preferably solid, with nucleic acid probes attached to the support. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., Science, 251:767-777 (1991). Each of which is incorporated by reference in its entirety for all purposes.

Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as optical fibers, glass or any other appropriate substrate. (See U.S. Pat. Nos. 5,770,358, 5,789,162, 5,708,153, 6,040,193 and 5,800,992, which are hereby incorporated by reference in their entirety for all purposes.)

Preferred arrays are commercially available from Affymetrix under the brand name GENECHIP® and are directed to a variety of purposes, including genotyping and gene expression monitoring for a variety of eukaryotic and prokaryotic species. (See Affymetrix Inc., Santa Clara and their website at affymetrix.com.) Methods for preparing sample for hybridization to an array and conditions for hybridization are disclosed in the manuals provided with the arrays, for example, for expression arrays the GENECHIP Expression Analysis Technical Manual (PN 701021 Rev. 5) provides detailed instructions for 3′ based assays and the GeneChip® Whole Transcript (WT) Sense Target Labeling Assay Manual (PN 701880 Rev. 2) provides whole transcript based assays. The GeneChip Mapping 100K Assay Manual (PN 701694 Rev. 3) provides detailed instructions for sample preparation, hybridization and analysis using genotyping arrays. Each of these manuals is incorporated herein by reference in its entirety.

An allele refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”. At each autosomal specific chromosomal location or “locus” an individual possesses two alleles, one inherited from one parent and one from the other parent, for example one from the mother and one from the father. An individual is “heterozygous” at a locus if it has two different alleles at that locus. An individual is “homozygous” at a locus if it has two identical alleles at that locus.

Polymorphism refers to the occurrence of two or more genetically determined alternative sequences or alleles in a population. A polymorphic marker or site is the locus at which divergence occurs. Preferred markers have at least two alleles, each occurring at frequency of preferably greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens. Single nucleotide polymorphisms (SNPs) are positions at which two alternative bases occur at appreciable frequency (>1%) in the human population, and are the most common type of human genetic variation.

The term genotyping refers to the determination of the genetic information an individual carries at one or more positions in the genome. For example, genotyping may comprise the determination of which allele or alleles an individual carries for a single SNP or the determination of which allele or alleles an individual carries for a plurality of SNPs. For example, a particular nucleotide in a genome may be an A in some individuals and a C in other individuals. Those individuals who have an A at the position have the A allele and those who have a C have the C allele. In a diploid organism the individual will have two copies of the sequence containing the polymorphic position so the individual may have an A allele and a C allele or alternatively two copies of the A allele or two copies of the C allele. Those individuals who have two copies of the C allele are homozygous for the C allele, those individuals who have two copies of the A allele are homozygous for the C allele, and those individuals who have one copy of each allele are heterozygous. The array may be designed to distinguish between each of these three possible outcomes. A polymorphic location may have two or more possible alleles and the array may be designed to distinguish between all possible combinations.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur at equal frequency, and linked locus Y has alleles c and d, which occur at equal frequency, one would expect the combination ac to occur at a frequency of 0.25. If ac occurs more frequently, then alleles a and c are in linkage disequilibrium. Linkage disequilibrium may result, for example, because the regions are physically close, from natural selection of certain combination of alleles or because an allele has been introduced into a population too recently to have reached equilibrium with linked alleles. A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a gene (including regulatory sequences) (Y) that is a causative element of a phenotype, can be detected to indicate susceptibility to the disease in circumstances in which the gene Y may not have been identified or may not be readily detectable.

Normal cells that are heterozygous at one or more loci may give rise to tumor cells that are homozygous at those loci. This loss of heterozygosity may result from structural deletion of normal genes or loss of the chromosome carrying the normal gene, mitotic recombination between normal and mutant genes, followed by formation of daughter cells homozygous for deleted or inactivated (mutant) genes; or loss of the chromosome with the normal gene and duplication of the chromosome with the deleted or inactivated (mutant) gene.

A homozygous deletion is a deletion of both copies of a gene or of a genomic region. Diploid organisms generally have two copies of each autosomal chromosome and therefore have two copies of any selected genomic region. If both copies of a genomic region are absent the cell or sample has a homozygous deletion of that region. Similarly, a hemizygous deletion is a deletion of one copy of a gene or of a genomic region.

Genetic rearrangement occurs when errors occur in DNA replication and cross over occurs between nonhomologous regions resulting in genetic material moving from one chromosomal location to another. Rearrangement may result in altered expression of the genes near the rearrangement.

An aneuploid is a cell whose chromosomal constitution has changed from the true diploid, for example, extra copies of a chromosome or chromosomal region.

An individual is not limited to a human being, but may also include other organisms including but not limited to mammals, plants, bacteria or cells derived from any of the above.

The Whole Genome Sampling Assay (WGSA) reduces the complexity of a nucleic acid sample by amplifying a subset of the fragments in the sample. A nucleic acid sample is fragmented with one or more restriction enzymes and an adapter is ligated to both ends of the fragments. A primer that is complementary to the adapter sequence is used to amplify the fragments using PCR. During PCR fragments of a selected size range are selectively amplified. The size range may be, for example, 400-800 or 400 to 2000 base pairs. Fragments that are outside the selected size range are not efficiently amplified.

The fragments that are amplified by WGSA may be predicted by in silico digestion and an array may be designed to genotype SNPs that are predicted to be amplified. Genotyping may be done by allele specific hybridization with probes that are perfectly complementary to individual alleles of a SNP. A set of probes that are complementary to the region surrounding each SNP may be present on the array. Perfect match probes are complementary to the target over the entire length of the probe. Mismatch probes are identical to PM probes except for a single mismatch base. The mismatch position is typically the central position so for a 25 base probe the mismatch is position 13.

Other methods of complexity reduction include, for example, AFLP, see U.S. Pat. No. 6,045,994, which is incorporated herein by reference, and arbitrarily primed-PCR (AP-PCR) see McClelland and Welsh, in PCR Primer: A laboratory Manual, (1995) eds. C. Dieffenbach and G. Dveksler, Cold Spring Harbor Lab Press, for example, at p 203, which is incorporated herein by reference in its entirety. Additional methods of sample preparation and techniques for reducing the complexity of a nucleic sample are described in Dong et al., Genome Research 11, 1418 (2001), in U.S. Pat. Nos. 6,361,947, 6,391,592, 6,458,530, 6,632,611, 6,958,225 and U.S. Patent application Nos. 20030036069, 20050260628, and 20040067493, which are incorporated herein by reference in their entireties.

The design and use of allele-specific probes for analyzing polymorphisms is described by e.g., Saiki et al., Nature 324, 163-166 (1986); Dattagupta, EP 235,726, Saiki, and WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles.

Strand Displacement Amplification (SDA). The isothermal technique of rolling circle amplification (RCA) has been developed for amplifying large circular DNA templates such as plasmid and bacteriophage DNA (Dean et al., 2001). Using phi29 (also referred to as φ29) DNA polymerase, which synthesizes DNA strands up to about 70 kb in length using random exonuclease-resistant hexamer primers, in a reaction that is incubated at a single temperature, for example, 30 to 37° C. Unlike PCR the reaction is not subjected to multiple cycles of heating to denature followed by extension. Secondary priming events occur on the displaced product DNA strands, resulting in amplification via strand displacement. A thermophilic polymerase may be used, but the polymerase may be a mesophilic polymerase. Phi29 and methods of using phi29 is described in U.S. Pat. Nos. 5,576,204, 5,198,543, 5,001,050, and Blanco, L. and Salas, M. (1984) Proc. Natl. Acad. Sci. USA, 81, 5325-5329, Blanco. L., et al. (1989) J. Biol. Chem., 264, 8935-8940, and Garmendia, C., et al. (1992) J. Biol. Chem., 267, 2594-2599. Whole genome amplification methods using phi29 are also disclosed in U.S. Pat. Nos. 6,977,148, 6617,137 and 6,323,009. Kits for whole genome amplification are available from GE Healthcare, the GenomiPhi DNA Amplification Kit, and from Qiagen, the REPLI-G® Kit. Other DNA polymerases that may be used include, for example, Tts DNA polymerase, phage M2 DNA polymerase, VENT™ DNA polymerase, Klenow fragment of DNA polymerase I, T5 DNA polymerase, PRD1 DNA polymerase, T4 DNA polymerase holoenzyme, T7 native polymerase T7 SEQUENASE®, or Bst DNA polymerase. In some embodiments of SDA two sets of primers may be used.

Multiple Displacement Amplification (MDA): The principles of RCA have been extended to WGA in a technique called multiple displacement amplification (MDA) (Dean et al., 2002; and U.S. Pat. Nos. 6,280,949 and 6,617,137). In this technique, a random set of primers is used to prime a sample of genomic DNA. By selecting a sufficiently large set of primers of random or partially random sequence, the primers in the set will be collectively, and randomly, complementary to nucleic acid sequences distributed throughout nucleic acids in the sample. Amplification proceeds by replication with a highly possessive polymerase, for example, phi29 DNA polymerase, initiating at each primer and continuing until spontaneous termination. Displacement of intervening primers during replication by the polymerase allows multiple overlapping copies of the entire genome to be synthesized.

(C) Methods for Genetic Analysis Using Whole Genome Hybridization (WGH)

In some aspects, methods are disclosed for analysis of genetic information using whole genome amplification using (WGA) followed by whole genome hybridization (WGH) to an array of probes. The methods vary from methods that involve a complexity reduction step, such as the whole genome sampling assay (WGSA) in that the present methods amplify the entire genome instead of a representative subset of the genome. The WGSA method includes a restriction digestion step, an adapter ligation step and a PCR amplification step, but WGH uses instead a WGA step using random primers and a phi29 DNA polymerase. A genomic sample subjected to WGA can be hybridized directly to a SNP genotyping array to obtain accurate genotype calls using allele specific hybridization of oligonucleotide probes. The call rate and accuracy of genotyping using WGA and WGH is comparable to methods that employ a complexity reduction amplification step prior to hybridization to the genotyping array.

In another aspect SNPs are screened to identify SNPs that can be genotyped using WGH. In one aspect a screening array that includes sets of allele specific probes that are complementary to one or the other allele of a biallelic SNP are hybridized with a genomic sample that has been amplified by WGA and labeled. The hybridization pattern is analyzed and base calling software is used to determine if a genotype call can be made for each SNP and for each sample. Many different samples are analyzed using the same array, preferably samples that have been genotyped at these SNPs using another genotyping method. The accuracy of the calls that are made is determined. If a SNP is called in at least a threshold number of samples with at least a threshold level of concordance with the known genotype of the sample then that SNP is identified as one that can be genotyped using WGH.

In one aspect 3 to 6 million SNPs may be screened to identify a subset of SNPs, preferably more than 1 million and more preferably more than 2 million that can be genotyped by WGH. In one aspect the screening is done using an array of genotyping probes as described in U.S. Patent Publication Nos. 20060024715 and 20050227244 and in U.S. patent application Ser. No. 11/406,880. Screening methods are also disclosed in U.S. Provisional patent application No. 60/699,438.

Methods for High-Specificity Amplification

Methods for amplification of nucleic acid using a mixture of primer sequences are disclosed. The mixture of primers is designed to minimize complementarity between primer sequences while maintaining complexity in the mixture of primers. The use of the multiple strand displacement reaction for amplification of genomic DNA is described in U.S. Pat. Nos. 6,280,949 and 6,977,148, US patent publication 20030143587, Dean et al. Proc. Natl. Acad. Sci. USA 99. 5261 (2002) and Hosono et al., Genome Res. 13, 954 (2003). Kits for multiple displacement amplification are available, for example, the Qiagen Repli-G kit. Details of the method are included in the instructions provided with the Repli-G kit. In general, double stranded high molecular weight genomic DNA is denatured, and random hexamers are used in extension reactions with a highly processive DNA polymerase (eg. phi29). This polymerase has a strong strand displacement activity which efficiently displaces any double stranded DNAs in its polymerization path. Such displaced DNAs are then available for yet another round of repetitive chain extension by the random hexamers. This reaction essentially cycles over and over until reagents run out, or the fragments become too small. Since the strand separation step is carried out by the polymerase, this reaction is isothermal in nature and therefore does not require the temperature cycling events required by PCR.

However, in this procedure, a high concentration of random-hexamer oligonucleotide primer is necessary for the reaction to occur. As a consequence, such random hexamers can aggregate to form concatamers which are then amplified along with the input DNA sample. This leads to a significant amount of non-specific, non-representative amplified DNAs that do not arise from the target intended for amplification. In reactions where no-input DNA is added to the reaction, a large amount of output DNA is still produced. In reactions where input DNA is put into the reaction, the non-specific portion of the output DNA is reduced, but could still be as high as 10 to 30% of the total output mass, depending on various factors affecting the reaction efficiency. While such “carry-over” DNAs may not have significant effects on downstream applications requiring a target specific amplification step, for example, assays that directly analyze the WGA product may be adversely affected.

In a preferred embodiment, a semi-random oligonucleotide is used in place of random-hexamers. Random-hexamers generally are a mixture of oligonucleotides consisting of any of the 4 possible DNA bases (A, C, G, and T) at any of the 6 given positions on the primer. The semi-random oligonucleotide is preferably a 6-mer composed entirely of two non-complementary bases, for example, A or G, T or C, T or G, or A or C. The semi-random oligonucleotide 6-mers are composed of only 2 out of the 4 possible bases and the bases are selected to be non-complementary so the primers do not contain regions of self-complementarity. Minimizing self-complementarity in the primers results in reduction or elimination in the ability of the oligonucleotides to concatemerize and serve as templates for non-specific DNA amplifications. Although the use of 2 bases will result in fewer positions in the genome that are complementary to the primers than with a completely random 6-mer, the long length of the polymerase produced DNAs (10 kb on average) minimizes or eliminates any amplification biases that can result from using semi-random primer mixtures. The concentrations of the primers may need to be empirically optimized, but all other reaction components and procedures may be identical to those used with random primers containing all 4 bases.

In another embodiment the primers are RNA. Duplexes formed between a first and a second RNA primer would be inefficiently extended by a DNA dependent DNA polymerase so primer concatamers would not form or would form inefficiently. The RNA primers would be extended when hybridized to the DNA target.

In preferred embodiments the amplified nucleic acid sample is fragmented prior to hybridization to locus or allele specific probes. Fragmentation methods include, for example, digestion with a non-specific nuclease such as DNase I, mechanical shearing, or treatment of a uracil containing amplification product with a uracil DNA glycosylase followed by fragmentation of resulting abasic sites by, for example, treatment with an AP endonuclease such as APE 1 or by chemical or heat treatment. Methods for fragmenting nucleic acids are disclosed for example in US Patent Publication Nos. 20050191682 and 20050123956. Labels and methods of labeling are discussed above.

In some embodiments the amplification product generated by the disclosed methods may be fragmented, labeled and hybridized to an array of oligonucleotide probes attached to one or more solid supports. The solid support may be, for example, a bead, membrane, glass slide or other solid support. The array preferably has more than 5,000, 10,000, or 100,000 different species of probes. In particularly preferred embodiments the array has more than 1,000,000 different species of probes or more than 5,000,000 different species of probes. The array preferably interrogates the genotype of more than 1,000, 10,000, 100,000, 500,000, 1,000,000 or 2,000,000 different polymorphisms. In a particularly preferred aspect the polymorphisms are single nucleotide polymorphisms (SNPs) and preferably human SNPs. Arrays and methods for genotyping by hybridization to high density oligonucleotide microarrays are disclosed in US Patent Publication Nos. 20040185475, 20040146890, 20030186279, 20060024715, and 20050227244. In some aspects the array may be an array of beads, for example, see US Patent Publication No. 20050059048.

In some aspects the amplified sample is analyzed by whole genome hybridization for methylation status of one or more CpGs. For a discussion of methods useful for analyzing methylation see US Patent Pub. Nos. 20050196792, 20050153347 and 20050009059.

In a preferred embodiment the amplification product is hybridized to a GENECHIP Mapping Array from Affymetrix. Such arrays include the Mapping 10K, Mapping 100K and Mapping 500K arrays and array sets. The hybridization may be done according to the manufacturer's instructions. For detailed methods see also Klein et al., Science 308:385-9 (2005), Matsuzaki et al., Nat Methods 1: 109-11 (2004), Di et al, Bioinformatics, 21:1958 (2005), Bonnen et al., Nat. Genet. 38:214-7 (2006) and Matsuzaki et al., Genome Res. 14:414-25 (2004). For methods of using mapping arrays for copy number analysis see, for example, Huang et al, Hum Genomics 1:287-99 (2004) and Bignell et al., Genome Res., 14:287-95 (2004).

In preferred aspects genomic DNA is amplified using semi-random primers (composed of A and G, C and T, A and C or G and T). The semi-random primers used for amplification are entirely composed of non-complementary bases so that no two primers in the semi-random primer solution are complementary. Using primers that are not capable of hybridizing to form primer hybrids minimizes the potential for primers to hybridize to and extend other primers and form concatamers of primers independent of the genomic DNA. The reduction of the template independent primer amplification products results in a complexity in the amplification product that is representative of the complexity of the starting sample. The complexity is thus reduced compared to amplification products primed by random primer compositions that include self complementary primers.

In another aspect whole genome amplified samples are directly hybridized to an array of probes. Methods for direct genomic DNA hybridization without reduction in complexity are disclosed. Historically, it has been difficult to hybridize a very complex sample, like a whole genome sample, to an array because the hybridization rate is slow and the background hybridization resulting from non- or semi-specific hybridization is higher, resulting in relatively lower signal to noise ratios. Methods to increase hybridization stringency and reduce cross-hybridization are disclosed.

In one aspect non-specific competitor is added to the hybridization to increase specificity. Specific probe-target interactions are enhanced by increasing the rate of hybridization or the time of hybridization. The non-specific competitor may be for example, random oligos of fixed lengths, COT1 DNA, COT1 RNA, human repeat blocking oligos, low/high complexity non-specific DNA, and RNA. US Patent Pub. No. 20070003938, which is incorporated herein by reference, discloses buffers and methods for hybridization of genomic nucleic acid to arrays without complexity reduction.

In one aspect known repetitive elements in genomic DNA can be blocked during the hybridization by the addition of oligonucleotide blockers that are complementary to specific repeats. Fractions of genomic DNA that are enriched for repetitive elements such as COT-1 DNA (Invitrogen) and Hybloc (Applied Genetics) are often used as non-specific competitor in hybridization reactions. Use of oligonucleotide sequences targeted to repeats may also be used. Repetitive elements that may be targeted include SINEs (Alu, MIR, MIR3), LINEs (LINE1, LINE2, LINE3), LTR elements (ERV Class I, ERV-(K) Class II, ERV-(L) Class III, MaLR), DNA elements (hAT group (MER1-charlie, Zaphod), Tc-1 group (MER2-Tigger, Tc2, Mariner), PiggyBac-like and Unclassified). See International Human Genome Sequencing Consortium, Nature 409:860-921 (2001).

In another aspect hybridization rate enhancers may be used. For example, hybridization accelerants such as CTAB and PERT may be used. Volume excluders such as dextran sulfate or polyethylene glycol may also be used. The pH or concentration of DNA target may be modified to increase hybridization. Physical agitation may be used.

To increase hybridization stringency different salts may be used at varied concentrations, for example, SSPE, SSC, TMACl, and phosphate. Different temperatures may be used in combination with different salts, for example, TEACl. TEACl is a stronger denaturant than TMACl and can be used to increase stringency using lower temperatures. See Nuc. Acids Res. (1988), 16(10):4637-50. Denaturants may be added, for example, formamide, DMSO, Betaine, glycerol, urea, or formaldehyde. In another aspect PVP is used in the whole genome hybridization reactions as a non-specific competitor to increase signal to noise.

Methods for selecting SNPs that can be accurately genotyped using the methods disclosed herein are also disclosed. Some SNPs show better performance depending on the amplification and genotyping method and may be selected for inclusion on a genotyping array and assay. In one aspect a large number of SNPs may be screened using a particular detection assay, for example, a genotyping array. Those SNPs that work reproducibly with the hybridization method can be selected for inclusion in a final assay. For example, 4 million SNPs may be screened using a particular whole genome hybridization method. If 50% of the SNPs are reproducibly called with the desired accuracy rate and concordance those 2 million can be used as a pool to select SNPs for analysis. An array can be made to interrogate only the SNPs or a subset of the SNPs that pass the screening. SNPs can be further selected from the group that pass the screening based on other considerations, for example, if two SNPs that are close together and expected to show very high LD are in the set one may be selected for the final assay. SNPs that are in genes may be given preference. SNPs that have high minor allele frequency may be selected. SNPs that are polymorphic in multiple populations may be selected.

Many of the genotyping arrays available rely on specific hybridization between target and short DNA probe, for example, the Affymetrix Mapping Arrays (10K, 100K set and 500K set) use allele specific 25 base probes for detection of genotypes. The performance of these arrays depends in part on the specificity of the hybridizations where the tiled perfect match and mismatch probes hybridizes to the target DNA in a specific manner. In a given mixture of target sequences, specificity of the hybridizations decreases with increasing sequence complexity of the labeled DNA target sequences. With increased sequence complexity, background hybridization of near-matches, or partial matches often contributes to a level of hybridization sufficiently significant to reduce the confidence of measurement from the specific readout of DNA molecules that are perfect matches. Complexity-reduction techniques may be used to decrease the overall sequence complexity in the target sample prior to hybridization. However, this adds extra steps to the procedure and can introduce biases and reduce the amount of information that can be analyzed.

One possible way to increase the specific signal on chips over that arising from background hybridization is to apply more stringent hybridization or chip washing conditions to reduce the amount of non-specific hybridization. The length and sequence composition of the DNA pairing on the hybridized probes determine the maximum stringency conditions that can be empirically applied. Therefore, longer DNA probes may potentially provide for opportunities to apply more stringent conditions with high sequence complexity targets. On the other hand, as the length of the base-pairing increases, the ability to discriminate single base changes at high resolution decreases because with increasing DNA length, Tm differences caused by a single base mismatch are reduced. Methods to substantially increase the Tm differences occurring between hybridizations of a perfect match from that of a single base mismatch probe on a longer (25 bp-60 bp) probe array by using cleavage assays on chips are disclosed.

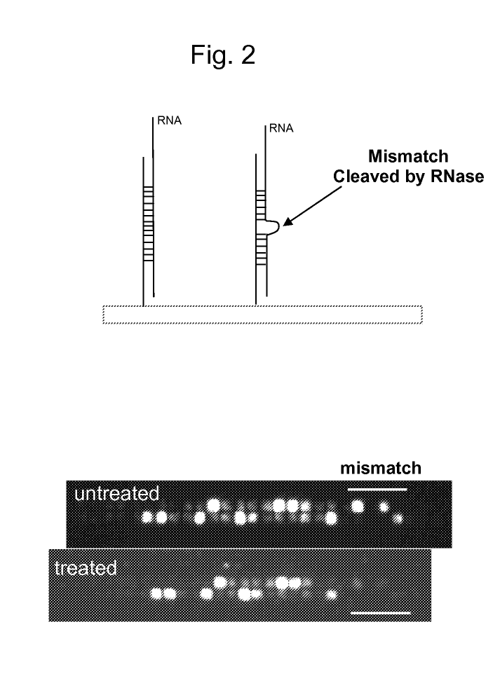

The method is a variation of the “RNA protection assay”, except in the described method the probe is a DNA molecule and reactions are performed with molecules immobilized on arrays. The sample to be genotyped (or sequenced) is first converted to RNA molecules using in vitro transcription with phage RNA polymerases. The transcribed RNA incorporates biotinylated ribonucleotides during synthesis. These biotin-labeled RNA molecules are fragmented and hybridized to arrays. The optimal length of the tiled probes on the arrays may be determined experimentally, although the hybridization temperature may be predicted computationally to facilitate protocol optimization. Once hybridized, the amount of hybridization to the perfect match probes and to the mismatch probes may be distinguished in order to determine the sequence identity of the base in question (usually the middle nucleotide in a given probe). This is accomplished by RNase treatment of the hybridized chip. Enzymes that may be used include: Rnase Ti, Al or Rnase One enzyme; or a combination of the enzymes listed. RNase is an enzyme that specifically degrades single stranded RNA. It does not have an effect on double stranded RNA molecules, or on RNA molecules hybridized to DNA except in regions of mismatch where one or more bases is left in a single-stranded state. In regions of mismatch, the RNAse enzyme will cleave the RNA molecule that is hybridized to the DNA, whereas on a perfect match hybrid, the RNA molecule is left intact. After the cleavage reaction, the chip is washed with a stringent buffer to remove the cleaved RNA molecules off the array. The biotin label on the intact RNA molecules can be detected via standard staining and scanning procedures. An alternative procedure is to label the hybridized RNA molecules with a fluorescent dye using polyA polymerase incorporation after the stringency wash. See FIG. 2.

In another aspect, mismatch cleaving enzymes may be used. Mismatch cleaving enzymes include, for example, SURVEYOR® CEL I Nuclease (Transgenomic, Inc.) and CEL I mismatch endonuclease (Oleykowski et al., Nucleic Acids Res. 26:4597-4602 (1998)). Other single strand specific nucleases may also be used, for example, mung bean nuclease and S₁ nuclease. See, Till et al., Nucleic Acids Res. 32:2632-2641 (2004).

For additional background on mismatch cleavage methods see Shenk et al., Proc. Natl. Acad. Sci. USA 72:989-993 (1975), Melton, D. A. et al. (1984), Nucl. Acids Res. 12, 7035, Sambrook, J., Fritsch, E. F. and Maniatis, T. (1989) Molecular Cloning: A Laboratory Manual, 2nd Edition, Cold Spring Harbor Laboratory Press, Cold Spring Harbor and Gilman, M. (1989) In: Current Protocols in Molecular Biology, Vol. 2, Ausubel, F. et al., eds., John Wiley and Sons, New York.

### EXAMPLES

### Example 1

Human HapMap DNA samples from 51 individuals were obtained from Coriell. For each of the 51 samples 20 μl (1 μg) template DNA was mixed with 20 μl buffer D1 (Qiagen Repli-G Midi Kit) and vortexed, spun and incubated for 3 min at room temp. 40 μl of Buffer NI (Qiagen) was added followed by vortexing and spinning. 312 μl of WGA MasterMix (Qiagen) and 8 μl of phi29 DNA polymerase (Qiagen) were added and the samples were incubated at 30° C. overnight and then at 65° C. for 10 min. The amplified samples were fragmented as follows: 400 μl of amplified DNA was mixed with 40 μl of 10× fragmentation buffer from the Affymetrix 500K Mapping Assay reagent kit and 2.5 μl of fragmentation reagent from the Affymetrix 500K Mapping Assay reagent kit. The samples were incubated at 37° C. for 12 min and at 95° C. for 15 min. The samples were then ethanol precipitated by addition of 40 μl of 3M NaOAc and 1000 μl of absolute ethanol. The samples were then resuspended in 135 μl water, 15 μl 10× fragmentation buffer (500K kit), 46 μl 5× TdT buffer (500K kit), 15 μl 30 mM DLR (500K kit) and 15 μl TdT (500K kit). The samples were incubated at 37° C. for 2 hours then 95° C. for 15 min. Samples were then ethanol precipitated by addition of 25 μl 3M NaOAc and 560 μl absolute ethanol and spun at 4° C. for 20 min. The samples were resuspended in 250 μl hybridization solution (2.9 M TMACl, 10 mM Tris pH 8.0, 0.001% Triton X-100, 0.1 mg/ml herring sperm DNA and 10% yeast RNA (10 mg/ml). The sample was incubated with the array using standard protocols for 60 hours at 50° C. and 20 rpm. Washing, staining and scanning was as described in the manual for the Mapping 500K array set. The arrays used were Sty1 SNP screening array similar in format to the GeneChip Human Mapping 250K Sty1 array available from Affymetrix.

Genotypes were determined using the DM algorithm see U.S. Patent Publication No. 20050123971) and the BRLMM algorithm (see U.S. Patent Application No. 60/744,002 and Rabbee and Speed, Bioinformatics 2006, 22:7-12). Call accuracy was determined by comparison to HapMap reference genotypes. The samples included 19 Caucasians, 16 Africans and 16 Asians. The array was the 500K Sty I screening array design 1 which has 5 μm features and 56 probes per SNP. The array interrogates the genotype of 113,296 human SNPs. 2 samples were excluded from the final analysis because of damaged arrays leaving 49 total scans for analysis.

For the genotype calling first pass a conservative cut-off was used with the following filters being applied: SNP call rate greater than or equal to 85%, MAF greater than 0.02 and pHW greater than 0.00001. Using these parameters for DM at 0. 1 the conversion rate was 0.2222, concordance to HapMap 19 reference genotypes was 0.9953, concordance for homozygotes was 0.9958 and for heterozygotes was 0.9937. For BRLMM at 0.15 conversion rate was 0.3312, concordance 0.9953, concordance homozygotes 0.9949 and concordance heterozygots was 0.9964. There were 24,414 SNP converted by both DM and BRLMM, 25,171 converted by DM with 757 unique to DM, and 37,544 converted by BRLMM with 13,130 unique to BRLMM.

The calls made by WGH were compared to calls made in the samples using Whole Genome Sampling Assay (WGSA). Of the 49 samples there were 45 that were analyzed by WGSA for a total of 45×113,296=5,098,320 calls. There were 3,489,381 cases where calls were made by both WGSA and WGH and 92.7% of these calls were identical (3,233,176).

### Example 2

Whole genome hybridization using non-specific competitors. Inclusion of poly(vinylphosphate) [PVP] in hybridization. PVPS was not tested because solutions made with TMACl resulted in the generation of a cloudy precipitate. Possibly, PVPS could be used in MES or SSC hybridizations without TMACl.

The results of inclusion of PVP in comparison with yeast RNA is shown in Table 1.

| | DM p = 0.05 call | % SNPs >0.9 | |
|---|---|---|---|
| | rate | conformance | Signal to Noise |
| 12% Yeast RNA | 43.38% | 75.84% | 3.59 |
| 94 ul 10% PVP | 42.69% | 70.09% | 1.86 |
| 25 ul 10% PVP | 26.80% | 63.65% | 1.85 |
| 4 ul 10% PVP | 43.38% | 68.54% | 1.72 |

Resuspend RNA from torula yeast to 500 milligrams per mL in water (50% solution). This can be RNA from Ambion Cat#7118, or Sigma #3629, but some preparations of yeast RNA tested were not soluble at this concentration. Prepare 200 to 400 μg of genomic DNA or DNA target amplified using WGA by fragmenting and labeling with DLR using TdT. Combine the labeled DNA target with the yeast RNA in hybridization buffer (2.9M TMACl, 0.01% TritonX-100, 10 mM Tris-HCl pH 8.0) and hybridize at 50° C. for 60 hrs. Wash and stain using the Affymetrix fluidics station and scan using the Affymetrix GSC3000 using GCOS software. Genotype calls determined using BRLMM.

### Example 3

Increase specificity of WGA reaction when using semi-random primers. The control N6 is a random hexamer mix including all 4 nucleotides (A, C, G and T), M₆ has primers containing only A and C, R₆ has only A and G, Y₆ has only C and T, K₆ has only T and G and ri6 is rG/rA/rU/rC. For example, M₆ is 5′-MMMMMM-3′ where M is either A or C. Panels A and B are identical except for the polymerase used. In panel A the enzyme used was REPLIPHI™ phi29 DNA Polymerase (0.1 μg/μl) from Epicentre Biotechnologies, for panel B the enzyme was phi29 from New England Biolabs. Background amplification (−DNA) was dramatically reduced in the M₆ and R₆ samples. The N₆ sample showed amplification in both the +DNA and −DNA reactions (lanes 1 and 7) and for both enzyme preps (panels A and B). M₆ and R₆ showed the highest level of amplification in the +DNA sample with no detectable amplification in the −DNA sample indicating that primers composed of only A and C or only A and G can be used for WGA amplification to reduce non-specific amplification. Amplification was observed in the −DNA sample for N₆, M₆, R₆, Y₆ and K₆ with the NEB phi29 (panel B, lanes 7-11) suggesting that there may be DNA contamination in the enzyme preparation. The primers were phosphorothioate modified and resistant to exonuclease activity. For the Epicentre enzyme the recommended buffer is 40 mM Tris-HCL (pH 7.5), 500 mM KCl, 100 mM MgCl₂, 50 mM (NH₄)₂SO₄ and 4 mM DTT. The buffer provided with the NEB enzyme is 50 mM Tris-HCl, 10 mM (NH₄)₂SO₄, 10 mM MgCl₂ and 4 mM DTT with pH 7.5 at 25° C. and they recommend that the reaction also have 200 μg/ml BSA added and 200 μM dNTPs. The incubations were overnight at 30° C. in the buffers and conditions recommended by the vendors for each enzyme respectively. Both reactions had dNTPs added.

### CONCLUSION

It is to be understood that the above description is intended to be illustrative and not restrictive. Many variations of the invention will be apparent to those of skill in the art upon reviewing the above description. By way of example, the invention has been described primarily with reference to the use of a high density oligonucleotide array, but it will be readily recognized by those of skill in the art that other nucleic acid arrays, other methods of measuring signal intensity resulting from genomic DNA could be used. The scope of the invention should, therefore, be determined not with reference to the above description, but should instead be determined with reference to the appended claims, along with the full scope of equivalents to which such claims are entitled.

What is claimed is:

1. A method of amplifying a plurality of target sequences from a complex nucleic acid target sample, the method comprising:
forming an amplification mixture by bringing into contact a set of primers, DNA polymerase, and the complex nucleic acid target sample, wherein the target sample comprises said plurality of target sequences, and wherein the set of primers comprises at least 20 different species of primers, wherein each species of primer is at least 5 bases long, each species of primer has a different sequence and all primers in the set consist of the same two non-complementary bases; and
incubating the amplification mixture under conditions that promote replication of the target sequences, wherein the target sample is not subjected to denaturing conditions, wherein replication of the target sequences results in replicated strands, wherein during replication at least one of the replicated strands is displaced from the target sequence by strand displacement replication of another replicated strand.

2. The method of claim 1 wherein the primers each contain at least one modified nucleotide that results in the primers being resistant to 3′-5′ exonuclease.

3. The method of claim 1 wherein the DNA polymerase is phi29 DNA polymerase.

4. The method of claim 1 further comprising fragmenting the replicated strands and labeling the resulting fragments using terminal deoxynucleotidyl transferase.

5. The method of claim 4 wherein the fragments are labeled with modified nucleotides selected from the group consisting of biotinylated nucleotides, fluorescent nucleotides, 5 methyl dCTP, BrdUTP, or 5-(3-aminoallyl)-2′-deoxyuridine 5′-triphosphates.

6. The method of claim 1 further comprising incubating the polymerase-target sample mixture under conditions that promote strand displacement.

7. The method of claim 1 wherein the conditions that promote replication of the target sequence are substantially isothermic.

8. The method of claim 1 wherein the two bases are selected from the following pairs of two bases: guanine and adenine, cytosine and thymine, adenine and cytosine and guanine and thymine.

9. The method of claim 1 wherein the pairs of two bases are guanine and uracil or cytosine and uracil.

10. A method of amplifying a target nucleic acid sequence, the method comprising:
bringing into contact a set of random ribonucleotide primers, a strand displacing DNA dependent DNA polymerase, and a target sample, and incubating the target sample under conditions that promote replication of the target sequence, wherein the target sample is not subjected to denaturing conditions, wherein replication of the target sequence results in replicated strands, wherein during replication at least one of the replicated strands is displaced from the target sequence by strand displacement replication of another replicated strand.

11. The method of claim 10 wherein the random ribonucleotide primers are 6 to 10 nucleotides in length.

12. The method of claim 10 wherein each primer species contains at least one 2′-O-methyl ribonucleotide.

13. The method of claim 10 wherein nucleic acids in the target sample are not separated from other material in the target sample.

14. A method for determining the genotype of a plurality of polymorphisms in a sample derived from genomic DNA, comprising:
(a) obtaining a high complexity amplification product by a method comprising amplifying a plurality of nucleic acid sequences in the sample by a complexity amplification method, the complexity amplification method comprising:
mixing the sample with DNA polymerase and a set of at least 20 different sequence primers wherein the primers in the set are at least 6 nucleotides in length and consist of the same two non-complementary bases; and
incubating the mixture under conditions that promote replication of nucleic acid sequences in the sample, wherein replication of the sequences results in replicated strands, wherein during replication at least one of the replicated strands is displaced from the target sequence by strand displacement replication of another replicated strand;
(b) fragmenting the high complexity amplification product;
(c) labeling the high complexity amplification product;
(d) hybridizing the high complexity amplification product to a plurality of allele specific probes to obtain a hybridization pattern; and
(e) determining the genotype of a plurality of polymorphisms based on the hybridization pattern.

15. The method of claim 14, wherein the two non-complementary bases are selected from guanine and adenine, cytosine and thymidine, adenine and cytosine, or guanine and thymidine.

16. The method of claim 14 wherein the primer set is selected from R₆, Y₆, M₆ and K₆.

17. The method of claim 14, wherein the two non-complementary bases are selected from guanine and uracil or cytosine and uracil.

18. The method of claim 14 wherein the primers are resistant to nuclease.

19. The method of claim 14 wherein yeast tRNA is included in step (d).

20. The method of claim 14 wherein PVP is included in step (d).
